(19) United States
(12) Patent Application Publication
(10) Pub. No.: US 2007/0225577 A1
(43) Pub. Date: Sep. 27, 2007

(54) System and Method for Providing Sensor Based Human Factors Protocol Analysis

(75) Inventor: Santosh Mathan, Minneapolis, MN (US)

Correspondence Address: HONEYWELL INTERNATIONAL INC., 101 COLUMBIA ROAD, P O BOX 2245, MORRISTOWN, NJ, 07962-2245, US

(73) Assignee: Honeywell International Inc., Morristown, NJ (US)

(21) Appl. No.: 11/463,949

(22) Filed: Aug. 11, 2006

Related U.S. Application Data
(60) Provisional application No. 60/777,919, filed on Mar. 1, 2006.

(51) Int. Cl. A61B 5/00; G01D 1/00

(52) U.S. Cl. 600/301; 702/127

(57) ABSTRACT

An analysis system comprises at least one behavioral sensor configured to obtain user interaction data, at least one state sensor configured to obtain user impact data, and at least one processing unit. The at least one processing unit is configured to receive the user interaction data from the at least one behavioral sensor and the user impact data from the at least one state sensor. The at least one processing unit is further configured to index the user interaction data with the user impact data.

### RELATED APPLICATIONS

This application claims the benefit under 35 USC 119(e) of prior provisional application No. 60/777,919, filed Mar. 1, 2006, which is incorporated herein in its entirety by reference.

### BACKGROUND

The ongoing transformation of businesses and other organizations, such as the military and government, into more efficient and productive units depends at least in part on the effective use of a broad range of technological tools. These tools have the potential to enable individuals to become more effective performers. However, as the history of technology driven work transformation demonstrates, new technologies can often impact human performance negatively. In order to exploit the benefits of new tools, it is vital that potential usability problems be identified and remedied well ahead of deployment. For example, developing systems in the context of rapid iterative design cycles will require efficient and effective human factors assessment techniques.

While a variety of evaluation tools can be used, current methods are limited either in terms of their cost, effectiveness, or in terms of the time required to apply them. Research indicates that empirical observation of users performing tasks is one of the most effective ways to identify potential Human Systems Integration (HSI) problems. Human Systems Integration (HSI) refers to the integration of human considerations into the design and support of new technology.

Empirical data of interest to HSI practitioners include think aloud protocol, overt behavioral actions, and performance related measures such as time on task and errors. For example, data from empirical evaluations including verbal protocol, behavioral actions such as key strokes and mouse clicks, and performance measures such as time on task and errors can provide a great deal of insight into aspects of a system design that contribute to user workload.

However, empirical HSI analysis techniques have substantial costs associated with them. Video, audio, and detailed system log protocol are very time consuming and costly to analyze. Skilled analysts have to step through long protocol segments in order to characterize user behavior and estimate associated workload. For example, it is widely accepted in the HSI community that at least three hours of analysis must be spent for every hour of protocol. Besides being time consuming, the subjectivity involved in the coding and interpretation process can introduce errors in the analysis. The inherent time and cost also constrains the number of subjects that can be used in HSI analyzes. Inferences based on small samples introduce another source of error in the conclusions that can be drawn on the basis of empirical data.

As a response to some of the difficulties associated with empirical assessment, the HSI community has developed a wide range of formal analysis techniques that rely on cognitive and task models to predict the impact of various system designs on human performance. However, the use of formal analytic methods in the context of complex systems requires a great deal of time from highly skilled practitioners. Complex modeling efforts require personnel with extensive knowledge of the system, domain, and computer science. The time and effort required to develop and maintain models limits the utility of these techniques in the context of rapid and iterative design cycles.

Similarly, usability inspection techniques are widely used as low cost alternatives to the methods mentioned above. Teams of evaluators systematically step through a system and note violations of sound HSI design practices, or design decisions that are incongruent with likely cognitive strategies. These techniques can be used in conjunction with fully functional systems, as well as low fidelity prototypes. However, usability inspection methods have been shown to identify only a small subset of problems identified through usability testing. The effectiveness of these techniques can be raised by adding trained evaluators. However, this can add to the expense of evaluations. Similarly, workload rating scales, while a low-cost evaluation option, may be compromised by subjectivity and the limits associated with retrospection. Additionally, workload rating scales only provide a summary measure and may not point to the specific features and episodes contributing to workload.

### SUMMARY

In one embodiment, an analysis system is provided. The analysis system comprises at least one behavioral sensor configured to obtain user interaction data, at least one state sensor configured to obtain user impact data, and at least one processing unit configured to receive the user interaction data from the at least one behavioral sensor and the user impact data from the at least one state sensor. The at least one processing unit is further configured to index the user interaction data with the user impact data.

### DRAWINGS

The present invention can be more easily understood and further advantages and uses thereof more readily apparent, when considered in view of the description of the embodiments and the following figures in which:

FIG. 1 is a high level block diagram depicting a human factors analysis system according to one embodiment of the present invention.

FIG. 2 is depicts an exemplary query interface according to one embodiment of the present invention.

FIG. 3 depicts a test user wearing behavioral and state sensors according to one embodiment of the present invention.

FIG. 4 is an exemplary diagram depicting different patterns of electroencephalograph activity.

FIG. 5 is an exemplary chart depicting detection of caridiac peaks and spectral decomposition of Heart Rate Variability (HRV) data.

FIG. 6 is an exemplary chart depicting differences in scanning pattern behavior for two levels of workload.

FIG. 7 is a flow chart showing a method of performing human factor analysis according to one embodiment of the present invention.

Like reference numbers and designations in the various drawings indicate like elements.

### DETAILED DESCRIPTION

In the following detailed description, reference is made to the accompanying drawings that form a part hereof, and in which is shown by way of illustration specific illustrative embodiments in which the invention may be practiced. These embodiments are described in sufficient detail to enable those skilled in the art to practice the invention, and it is to be understood that other embodiments may be utilized and that logical, mechanical and electrical changes may be made without departing from the scope of the present invention. It should be understood that the exemplary methods illustrated may include additional or fewer steps or may be performed in the context of a larger processing scheme. Furthermore, the methods presented in the drawing figures or the specification are not to be construed as limiting the order in which the individual steps may be performed. The following detailed description is, therefore, not to be taken in a limiting sense.

Instructions for carrying out the various process tasks, calculations, and generation of signals and other data used in the operation of the systems and methods of the invention can be implemented in software, firmware, or other computer readable instructions. These instructions are typically stored on any appropriate computer readable medium used for storage of computer readable instructions or data structures. Such computer readable media can be any available media that can be accessed by a general purpose or special purpose computer or processor, or any programmable logic device.

Suitable computer readable media may comprise, for example, non-volatile memory devices including semiconductor memory devices such as EPROM, EEPROM, or flash memory devices; magnetic disks such as internal hard disks or removable disks (e.g., floppy disks); magneto-optical disks; CDs, DVDs, or other optical storage disks; nonvolatile ROM, RAM, and other like media. Any of the foregoing may be supplemented by, or incorporated in, specially-designed application-specific integrated circuits (ASICs). When information is transferred or provided over a network or another communications connection (either hardwired, wireless, or a combination of hardwired or wireless) to a computer, the computer properly views the connection as a computer readable medium. Thus, any such connection is properly termed a computer readable medium. Combinations of the above are also included within the scope of computer readable media.

In embodiments of the present invention, data obtained from cognitive and/or physical state sensors is classified by a processing unit and used to index behavior protocol (e.g. video streams, audio, keylogs, etc.) indicating a user's interaction with a technology or system under test. Hence, embodiments of the present invention, provide a human factors analyst with a way to identify segments of behavioral protocol (such as video and audio) that could point to the sources of usability problems with a system and significantly reduce the subjectivity, time, and cost associated with human factors analysis of test systems. Currently, a typical human factors analyst steps through long protocol segments (such as audio and video) in order to characterize user behavior and estimate associated workload based on inferences derived from observable errors, utterances, and actions. Not only are these inferences often time consuming to make, they are also subjective and potentially error prone.

Embodiments of the present invention substantially eliminate the time consuming, and error prone process of interpreting and coding behavioral protocol as well as enable quicker access to relevant segments of behavior protocol. By using sensor based estimates to index behavioral protocol, analysts are able to focus their efforts on examining protocol segments indexed with unusually high workload values. Focused analysis of protocol segments identified using sensor based workload metrics can help the human factors analyst pinpoint problematic aspects of a system quickly (e.g. within minutes of an evaluation session—instead of the long lags that typically characterize post hoc analysis). In addition, usability analysis based on neurophysiological and physiological sensors provides a more direct and less subjective estimate of the impact of a system on a user. By reducing the time, cost and subjectivity, analysts are also able to conduct more studies with more test users which increases the accuracy of the analysis. Hence, embodiments of the present invention enable the deployment of usable systems to the market or field in a more time and cost effective manner.

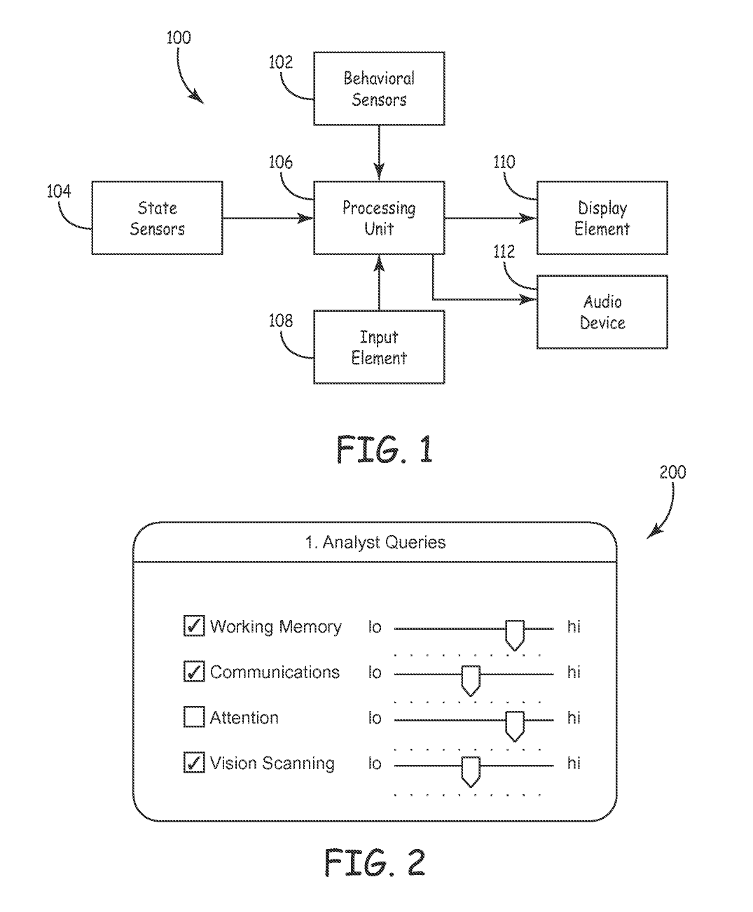

FIG. 1 is a high level block diagram depicting a human factors analysis system 100 according to one embodiment of the present invention. System 100 includes one or more behavioral sensors 102 and one or more state sensors 104. State sensors 104 can be congnitive and/or physical state sensors. Behavioral sensors 102 collect data regarding a user's interaction with a test system (not shown). In particular, behavioral sensors 102 collect, among other things, audio recordings, video screen captures, and point of view video streams, to provide an analyst with information about the test system state and the underlying task context. Examples of behavioral sensors 102 include, but are not limited to, a video camera, a still camera, an audio recorder, and a keystroke logger. As used herein, the data collected by behavioral sensors 102 is referred to as “user interaction data.” User interaction data, therefore, refers to data regarding overt physical actions of a user, such as keystrokes, mouse clicks, verbal utterances, etc. Hence, behavioral sensors 102 record what a user does in interacting with a test system so that the overall task context is accurately and thoroughly characterized by a variety of complementary techniques. Various configurations of different behavioral sensors 102 are used in operational settings, depending on the application contexts.

State sensors 104 collect data regarding the impact of the test system on a user. As used herein, the data collected by state sensors 104 is referred to as “user impact data.” User impact data, therefore, refers to data regarding the processing requirements and workload impact of a test system on users, such as data indicative of working memory, attention, and physical demands on a user. Examples of state sensors 104 include, but are not limited to, an electroencephalograph (EEG) sensor, an electrocardiogram (ECG) sensor, an eye motion detector, a head tracker, near infrared imaging sensor, and a dead reckoning module that tracks a user's physical position using a combination of a gyroscope and global positioning system (GPS) sensors. Hence, state sensors 104 collect objective data regarding a user's physiological, neurophysiological, and physical state while interacting with the test system. Various configurations of different state sensors 104 are used in operational settings, depending on the application contexts.

In this example, behavioral sensors 102 and state sensors 104 utilize a wearable and mobile form factor which enables workload estimation in a diverse range of contexts in which systems are actually used. For example, in a military setting, workload estimation using a wearable and mobile form factor is possible from aircraft and shipboard settings to dismounted operations. An exemplary mobile sensor suite is Honeywell's mobile sensor suite, developed under funding from the Defense Advanced Research Projects Agency and the US Army which has been validated in a variety of contexts—from stationary lab environments to military exercise events.

Behavioral sensors 102 and state sensors 104 communicate with a processing unit 106, in this example, via wireless links. However, in other embodiments, other means are used to provide user impact data and user interaction data to processing unit 106. For example, in other embodiments, processing unit 106 is coupled to behavioral sensors 102 and state sensors 104 via wired links, or the user interaction data and user impact data is saved in computer readable media such as DVDs, CDs, flash ROM, etc, for retrieval later by processing unit 106 located remotely.

Processing unit 106 is adapted to index the user interaction data with the user impact data received from behavioral sensors 102 and state sensors 104, respectively. In particular, in this example, processing unit 106 synchronizes the timing of the user interaction data and the timing of the user impact data so that it is possible to relate sensor based workload ratings with protocol segments. An analyst is then able to use an input element 108 to perform queries on the indexed user interaction data. For example, a user can query all segments where the frequency in the frontal region of the brain, as indicated by an EEG, is above a certain threshold or number of standard deviations above a norm. In this way the user is able to focus on only those segments which meet the specified criteria rather than spending time on segments which are not of interest.

In addition, in this example, processing unit 106 also relies on a set of signal processing components and a variety of machine learning techniques to correct for errors and/or noise, and to estimate workload on the basis of statistical regularities associated with different cognitive and physical states. For example, signals from body worn sensors are affected by a range of artifacts. These artifacts can often be more prominent than the signal of interest and are of particular concern in mobile environments where a user's movements may be relatively unconstrained. Processing unit 106 analyzes the statistical properties of artifacts. Once the regularities of noise sources are identified, processing unit 106 uses a variety of different techniques to boost the signal to noise ratio in the data. For example, a signal received from an EEG may contain noise spikes introduced by physical movement of the EEG and other sources of noise, such as a test user rubbing his/her eyes. In this example, with EEG signals, processing unit 106 uses band pass filters to eliminate DC drift and reduce the effects of electrical line noise. In addition, processing unit 106 performs adaptive linear filtering to correct for noise induced by eye blink activity. Processing unit 106 is also adapted to identify and reject segments affected by muscle activity. Muscle activity is identified, for example, by measuring high frequency content in the EEG spectra.

Processing unit 106 also classifies the user impact data and indexes the user interaction data using the classified user impact data. Hence, analysts are able to rapidly access segments of protocol associated with high workload values in order to identify likely usability problems. Exemplary signal processing components and machine learning techniques used to index the user interaction data using the user impact data include, but are not limited to, statistical analysis, thresholds, and classifiers. For example, a threshold may be set for a given measured attribute, wherein exceeding the threshold indicates a high workload. Similarly, a median value is determined, in one embodiment, for a given measured value and high workloads are determined based on the number of standard deviations from the median. All processing and logging of data occurs on a single computer, in some embodiments, so that system 100 can be used flexibly in a diverse range of operational settings.

Processing unit 106 also uses classifiers in some embodiments. For example, a plurality of classifiers are used to classify the user impact data as representative of high or low demands on attention levels, working memory, scanning effort, communication load, and the like. Classifiers include a variety of linear and non-linear pattern recognition techniques used in state classification and estimation to make robust inferences about cognitive state. These estimates can be either continuous workload values, or discrete estimates of workload classes (e.g. high vs. low workload) depending on the analysis requirements.

The classifiers used in processing unit 106 are feature selection algorithms used to identify specific features of the user impact data that are related to workload (e.g. working memory, scanning effort, etc.). By relying on a discriminative subset of features, processing unit 106 is better able to generalize and provide faster system performance. Processing unit 106, in some such embodiments, uses a committee of classifiers to decide on the most appropriate classification for each feature vector. A feature vector is a set of user impact data from one or more state sensors 104. Each classifier has its own set of strengths and weaknesses. By using a committee of classifiers, embodiments of the present invention allow the strengths of each classifier to be exploited, while minimizing their relative weaknesses.

Each classifier outputs a decision about every incoming feature vector. The class assigned to each feature vector is the majority vote of the committee of classifiers. Processing unit 106 then utilizes a modal filter to smooth the output of the committee of classifiers. Processing unit 106 analyzes the output of the committee of classifiers over a temporal window of several hundred milliseconds or seconds and determines cognitive state based on the modal state over the duration of the chosen window. That is, processing unit 106 analyzes the smoothed output to classify the test user workload at a given time based on the user impact data from different state sensors 104. Classification of the user impact data and indexing of the user interaction data with the classified user impact data, enables an analyst to perform weighted classification queries. For example, an analyst can query segments classified as high or low periods of working memory demands, scanning effort, etc.

Processing unit 106 is also coupled to an input element 108, in this example. Input element 108 enables analysts to enter dynamic queries to search for segments that point to potential usability problems. Based on the results of the query, processing unit 106 selects and retrieves segments of the indexed user interaction data and presents the segments to a user on a display element 110 and through an audio device 112. Display element 110 includes any display element suitable for displaying the various symbols and information for the operation of embodiments of the present invention. There are many known display elements that are suitable for this task, such as various CRT, active and passive matrix LCD, organic LED, and other existing or later developed display technology. Similarly, audio device 112 includes any audio device suitable for generating the various sounds and audio cues necessary for the operation of embodiments of the present invention. Alternatively, in other embodiments, processing unit 106 outputs segments of the indexed user interaction data to an electronic file stored on a computer readable-medium for later use by an analyst.

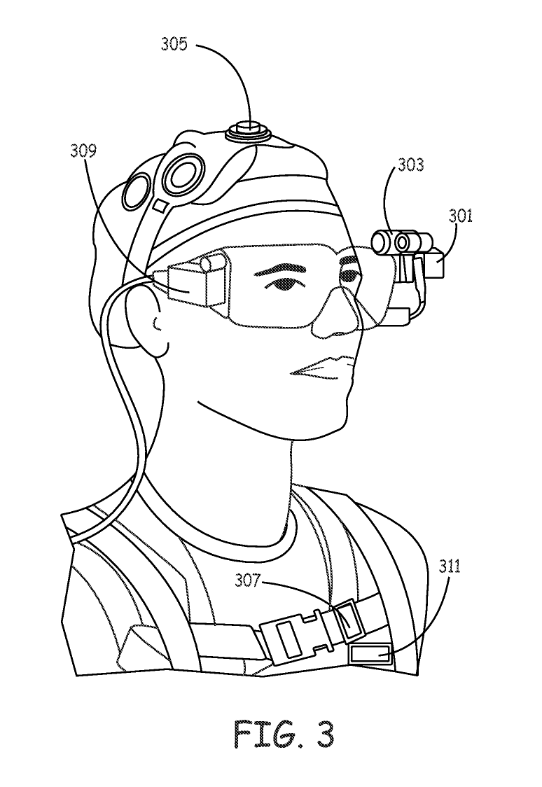

In operation, a test user (not shown) interacts with a test system. Exemplary test systems include but are not limited to, flight decks, computer programs, graphical user interfaces, communication systems, and the like. The test user is monitored by state sensors 104 and behavioral sensors 102. In some embodiments, the test user is wearing state sensors 104 and behavioral sensors 102 as shown in FIG. 3. In other embodiments, one or more behavioral sensors 102 and/or state sensors 104 are not worn by the test user. For example, a video camera may record the test user from a remote location. Processing unit 106 receives the user interaction data from behavioral sensors 102 and the user impact data from state sensors 104.

Processing unit 106 indexes the user interaction data with usability metrics based on user impact data provided by state sensors 104. An analyst then performs dynamic queries on the indexed user interaction data using input element 108 to identify behavioral protocol segments that point to potential usability problems. Dynamic queries enable analysts to incrementally adjust query criteria, such as with sliders 202 as shown in the exemplary query interface in FIG. 2, and view updates of query results on display element 110. Dynamic queries also enable users to filter information in complex information spaces very efficiently. Rapid and efficient analysis of behavioral protocol based on workload indexed criteria enables analysts to identify potential usability problems within a system very efficiently. For example, the analyst is able to focus on relevant segments and make note of aspects of the interaction between the test user and the test system when workload values deviate substantially from a normal range rather than spending large amounts of time reviewing irrelevant segments where the workload values are normal. In some embodiments, the analyst also shows some of the video segments to test users immediately following the test to solicit retrospective feedback. The feedback serves to validate the analyst's conclusions.

In addition, system 100 enables baseline comparisons across test systems by means of a variety of summary statistics associated with workload indices provided by processing unit 106. Summary statistics provide a gross indication of the usability impact of a test system as a whole on users. Immediately following the experimental session, the analyst is able to compare two or more systems based on summary statistics displayed on display element 110. For example, comparison of summary statistics helps identify test systems where average working memory load is significantly higher than in other test systems.

While the summary statistics suggest usability problems in a given test system, they do not point to a specific cause for the observed workload increase. However, system 100 enables the analyst to turn to a timeline view on display element 110 to learn more about potential design problems. For example, this timeline view provides a timeline representation of the point of view video captured over the course of the test session. The analyst is able to choose criteria for dynamic queries such as working memory load to identify problematic features. Processing unit 106 then provides the relevant segments to display element 110 based on the query results, as mentioned above.

FIG. 3 depicts a test user wearing behavioral and state sensors according to one embodiment of the present invention. The exemplary behavioral sensors and state sensors shown in FIG. 3 are examples of sensors described above with regards to behavioral sensors 102 and state sensors 104 in FIG. 1. In particular, the behavioral sensors shown in FIG. 3 include a microphone 301 and a video camera 303 and the state sensors include EEG sensor 305, ECG sensor 307, head tracker 309, and dead reckoning sensor 311.

A human factors analysis system, such as system 100, can utilize most commercial EEG systems both wired and wireless. However, in this example, a wireless EEG sensor 305 is used. The lack of wires reduces the potential for artifacts associated with the movement of wires in mobile environments. However, it is to be understood that a wireless EEG sensor is not required and wired EEG systems can be used in other embodiments. Data from EEG sensor 305 is spectrally decomposed to derive features that permit inferences about a user's cognitive state. Spectral analysis of EEG data permits inferences about the impact of complex tools on task relevant cognitive resources such as working memory and attention.

For example, working memory is a component of the human cognitive system that serves as a buffer for storing and managing information required to carry out complex cognitive tasks. Dynamic assessments of working memory load can serve as an indicator of the information processing demands imposed by a test system. High working memory demands can limit the ability of an individual to perform complex tasks effectively. Large working memory demands can also limit the ability of individuals to retrieve and encode task relevant information. This can lead to errors and may compromise situational awareness. Real-time indices of working memory provide a basis for comparing the workload impact of alternative test systems. Working memory indices also help identify specific design features that place high information processing demands within a particular test system.

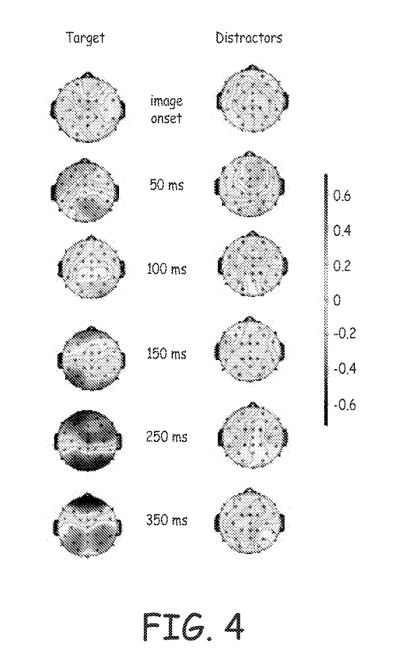

Research indicates that increases in working memory demands contribute to an increase in frontal midline theta and a decrease in parietal alpha. Embodiments of the present invention utilize EEG classifiers in a processing unit, such as processing unit 106 in FIG. 1, that have been able to classify working memory load with an accuracy of approximately 83% at temporal resolutions ranging from a few hundred milliseconds to under five seconds. Additionally, Evoked Response Potentials (ERP), a morphological change in EEG waveforms in response to task-relevant stimuli, are also used to estimate working memory demands. Using ERPs involves asking test users to attend to an auditory probe as they perform tasks using the test system under evaluation. The capacity to attend to the probe will vary inversely with increases in working memory demands. As can be seen in FIG. 4, the pattern of EEG activity of a test user varies based on the presence of a task relevant stimulus or a distractor. User impact data, such as the EEG activity patterns shown in FIG. 4, are provided by EEG sensor 305 to the processing unit. The processing unit utilizes detection algorithms (i.e. classifiers) to detect different patterns of activity. For example, in one embodiment, the processing unit uses single-trial ERP detection algorithms developed by Honeywell Inc. that have been shown to detect ERP patterns with an accuracy of approximately 90%.

Additionally, attention acts as the interface between the task environment and the cognitive system. Attentional resources help a user focus selectively on a particular channel of information, sustain that focus over time, and shift it appropriately in response to task demands. The impact of a system on human attention levels can be estimated using EEG sensor 305. For instance, increases in spectral power at 4 Hz and 14 Hz in midline sites accompany periods of low alertness. These changes can be used to reliably classify periods of low attention. Also, in some embodiments of the present invention, the NASA Engagement Index for use as a real-time index of attention is used by the processing unit in processing the user impact data from EEG sensor 305.

Workload measures derived from ECG sensor 307 are another element of the human factors analysis system, such as system 100. ECG sensor 307 complements EEG sensor 305, in one embodiment, and is used independently as the primary means for cognitive state estimation, in other embodiments. ECG based measures of workload serve to provide estimates of a user's stable cognitive state over relatively long windows of time. Unlike EEG sensor 305, which provides insight into cognitive state at temporal resolutions of milliseconds, ECG sensor 307 provides an assessment of cognitive state over the span of tens of seconds or minutes.

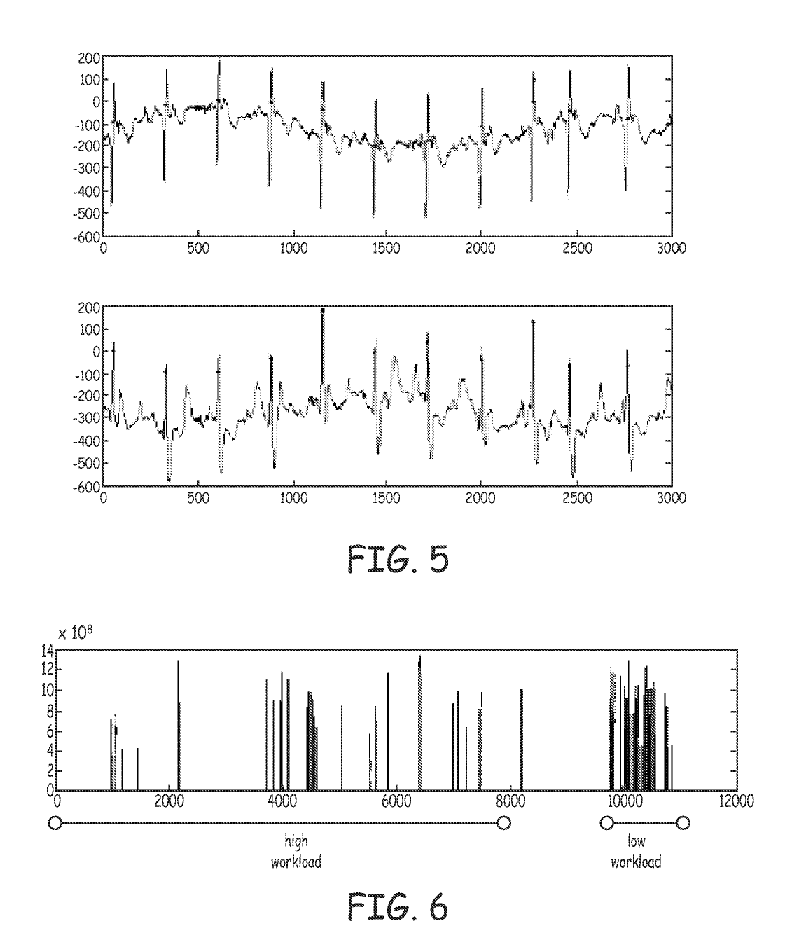

Metrics derived from spectral analysis of heart rate variability (HRV) data are used to estimate user workload. Research suggests that spectral power in the 0.05 Hz to 0.15 Hz range, reflects short term blood pressure regulation. Power in this spectral band bears an inverse relationship to workload. Embodiments of the present invention, in the context of working memory tasks, are able to discriminate between high and low working memory load at accuracies ranging from 77% to 91% for windows of less than 1 minute, depending on the subject. In contrast, most HRV based cognitive state estimation efforts rely on analysis windows spanning five minutes or more, and generally do not attempt to discriminate between workload levels—comparisons are typically between high workload and rest. FIG. 5 is an exemplary chart depicting detection of caridiac peaks and spectral decomposition of HRV data based on user impact data received by the processing unit from ECG sensor 307, in one embodiment of the present invention.

In addition, head tracker 309 and dead reckoning sensor 311 are used in this example to track physical correlates of workload. For example, aviation researchers have used heads-down time as a metric for assessing crew workload. The underlying assumption behind this metric is that the more difficult a test system is, the more attention it demands from users—leaving less time to scan outside the aircraft. In other contexts, the demands associated with the use of some test systems may induce a high degree of course variability as a user traverses a route by foot. Similarly, some systems may require a high degree of visual scanning to integrate information from spatially distributed displays, while others consolidate task relevant information within a single display. As these example situations illustrate, the demands associated with the use of a test system often place constraints on physical movement. Examining patterns of physical movement could provide insight into workload.

Head tracker 309, in this example, is a head tracker manufactured by InterSense Inc. and dead reckoning sensor 311 is a Honeywell Inc. Dead Reckoning Module (DRM). However, it will be understood that other head tracker and dead reckoning sensors can be used in other embodiments. Head tracker 309 samples head orientation around the yaw, pitch, and roll axes using an internal gyroscope. In this example, the sample rate is approximately 185 Hz. FIG. 6 provides an example of differences in scanning pattern behavior in two levels of workload detected by a head tracker such as head tracker 309. As can be seen in FIG. 6, high workload periods are characterized by a lower proportion of detected visual scanning than low workload periods.

Dead reckoning sensor 311, in this example, is a self contained navigation component that fuses information from several internal sensors to determine displacement from a specific geographical position. Exemplary internal sensors include but are not limited to, a thermometer, barometer, magnetometer, accelerometer, gyroscope, and GPS receiver.

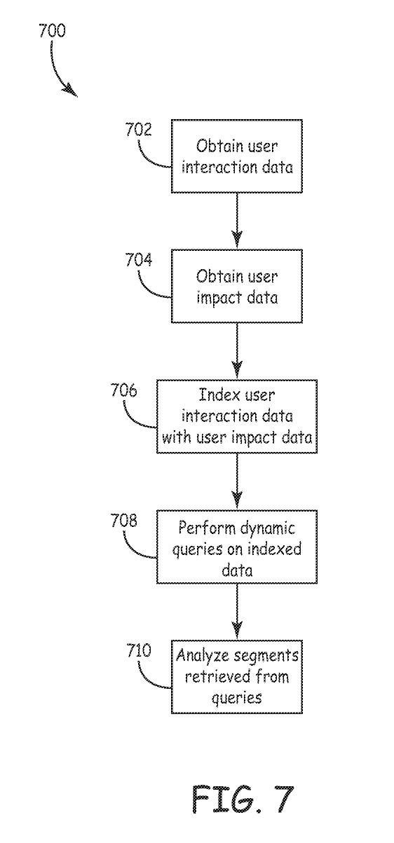

FIG. 7 is a flow chart showing a method 700 of performing human factor analysis according to one embodiment of the present invention. At block 702, user interaction data is obtained by one or more behavioral sensors, such as behavioral sensors 102 in FIG. 1, and received by a processing unit, such as processing unit 106 in FIG. 1. At block 704, user impact data is obtained by one or more state sensors, such as state sensors 104 in FIG. 1, and received by the processing unit. At block 706, the processing unit indexes the user interaction data with the user impact data, as described above. In particular, the processing unit synchronizes the user interaction data with the user impact data. In addition, the processing unit classifies segments of the user impact data using one or more classifiers to identify specific features (e.g. classifications) that are related to workload such as attention levels, working memory, scanning effort, communication load, and the like. In some embodiments, the classification of each segment is based on a majority vote of the one or more classifiers.

At block 708, an analyst performs dynamic queries on the indexed user interaction data. For example, an analyst may use a slider in a graphical user interface, such as query interface 200, in performing dynamic queries. Performing dynamic queries includes querying the indexed user interaction data corresponding to classifications of the user impact data. In addition, performing dynamic queries includes, in some embodiments, querying summary statistics generated for two or more test systems by the processing unit based on the user impact data from each of the two or more test systems. At block 710, the analyst is able to focus on the relevant segments of the user interaction data that is retrieved based on the dynamic queries. This eliminates the need for the analyst to spend time on segments that do not help determine potential usability problems. Hence, the analyst is able to more effectively analyze test results, which lowers the costs and time to market.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention. Therefore, it is manifestly intended that this invention be limited only by the claims and the equivalents thereof.

What is claimed is:

1. An analysis system, comprising:
at least one behavioral sensor configured to obtain user interaction data;
at least one state sensor configured to obtain user impact data; and
at least one processing unit configured to receive the user interaction data from the at least one behavioral sensor and the user impact data from the at least one state sensor, the at least one processing unit further configured to index the user interaction data with the user impact data.

2. The analysis system of claim 1, wherein the at least one behavioral sensor comprises at least one of a video camera, a still camera, an audio recorder, or a keystroke logger.

3. The analysis system of claim 1, wherein the at least one state sensor comprises at least one of an electroencephalograph sensor, an electrocardiogram sensor, an eye motion detector, a dead-reckoning sensor, or a head tracker.

4. The analysis system of claim 1, wherein the at least one behavioral sensor and the at least one state sensor are configured in a wearable and mobile form factor.

5. The analysis system of claim 1, wherein the at least one behavioral sensor and the at least one state sensor are coupled to the at least one processing unit via a wireless link.

6. The analysis system of claim 1, further comprising:
an input element configured to provide analyst queries to the at least one processing unit; and
a display element coupled to the at least one processing unit and configured to display segments identified by the at least one processing unit based on the analyst queries.

7. The analysis system of claim 1, wherein the at least one processing unit is further adapted to correct for errors and noise in the user impact data.

8. The analysis system of claim 1, wherein the at least one processing unit is further adapted to classify segments of the user interaction data to identify features that are related to workload.

9. The analysis system of claim 8, wherein the at least one processing unit uses a plurality of classifiers to classify segments of the user interaction data, wherein each classifier votes on the classification of the segments and a majority vote determines the classification for each segment of the user interaction data.

10. A computer program product comprising:
a computer-usable medium having computer-readable code embodied therein for configuring a computer processor, the computer-readable code comprising:
first executable computer-readable code configured to cause a computer processor to index user interaction data using corresponding user impact data; and
second executable computer-readable code configured to cause a computer processor to output indexed segments of the user interaction data corresponding to a user query.

11. The computer program product of claim 10, wherein the first executable computer-readable code is further configured to cause a computer processor to run a plurality of classifiers, each classifier adapted to analyze segments of the user impact data, and vote on the classification of each segment, wherein a majority vote of the plurality of classifiers determines each segment's classification.

12. The computer program product of claim 11, wherein the second executable computer-readable code is further configured to output indexed segments corresponding to weighted classification queries.

13. The computer program product of claim 10, further comprising third executable computer-readable code configured to cause a computer processor to analyze the user impact data, and output summary statistics regarding average workload of a test system based on the impact data.

14. The computer program product of claim 10, wherein the second executable computer-readable code is configured to output indexed segments of the user interaction data based on at least one of threshold queries or statistical queries.

15. The computer program product of claim 10, further comprising third executable computer-readable code configured to correct for errors and noise in the user impact data.

16. A method of performing human factor analysis, the method comprising:
obtaining user interaction data with one or more behavioral sensors;
obtaining user impact data with one or more state sensors;
indexing the user interaction data with the user impact data;
querying the indexed user interaction data to obtain one or more segments indicating potential usability problems; and
analyzing the obtained one or more segments.

17. The method of claim 16, wherein querying the indexed user interaction data further comprises querying summary statistics generated for two or more test systems based on the user impact data from each of the two or more test systems.

18. The method of claim 16, wherein indexing the user interaction data with the user impact data further comprises synchronizing the user interaction data and the user impact data.

19. The method of claim 16, wherein indexing the user interaction data with the user impact data further comprises classifying a plurality of segments of the user impact data using one or more classifiers.

20. The method of claim 19, wherein classifying the plurality of segments of the user impact data using one or more classifiers further comprises classifying each segment of the user impact data based on a majority vote of the one or more classifiers.
